SEQUENCE LISTING (6 sequences)

SEQ ID NO: 1 - DNA, 960 bp, Homo Sapiens
agtaggggcccgggcggaggcggtggcgggatggggctgctgctcatgatcctggcgtcggccgtgctgggttccttcctcacgctcctcgcccagttcttcctgctgtaccgcagacagcccgagccgccggcggacgaggccgcccgcgcgggcgagggcttccgctacatcaagccagtgccgggcctgctcctaagggagtacctttatggcggcggccgggatgaggagccctccggagcggcccctgagggcggcgcgacccccaccgcggcccccgagacccccgccccgccgacgcgggagacttgctacttcctcaacgccaccatcctattcctgttccgggagttgcgggacaccgcgctgacccgccgctgggtcaccaagaagatcaaggtggagttcgaggagctgctgcagaccaagacggccgggcgcctgctggaggggctgagcctgcgggacgtgttcctgggcgagacggtgcccttcatcaagaccatccggctcgtgcggccagtcgtgccctcggccaccggggagcccgatggccctgaaggggaggcgctgcccgccgcctgccccgaggagctggccttcgaggcggaggtggagtacaacgggggcttccacctggccatcgacgtggacctggtcttcggcaagtccgcctacttgtttgtcaagctgtcccgcgtggtgggaaggctgcgcttggtctttacgcgcgtgcccttcacccactggttcttctccttcgtggaagacccgctgatcgacttcgaggtgcgctcccagtttgaagggcggcccatgccccagctcacctccatcatcgtcaaccagctcaagaagatcatcaagcgcaagcacaccctaccgaattacaagatcaggtgagctggaggtcggggagggggcctggctgccgggaacccgggcctgggcgggacg

SEQ ID NO: 2 - DNA, 261 bp, Homo Sapiens
gatcaaggtggagttcgaggagctgctgcagaccaagacggccgggcgcctgctggaggggctgagcctgcgggacgtgttcctgggcgagacggtgcccttcatcaagaccatccggctcgtgcggccagtcgtgccctcggccaccggggagcccgatggccctgaaggggaggcgctgcccgccgcctgccccgaggagctggccttcgaggcggaggtggagtacaacgggggcttccacctggccatcgacgtgga

SEQ ID NO: 3 - DNA, 873 bp, Homo Sapiens
atggggctgctgctcatgatcctggcgtcggccgtgctgggttccttcctcacgctcctcgcccagttcttcctgctgtaccgcagacagcccgagccgccggcggacgaggccgcccgcgcgggcgagggcttccgctacatcaagccagtgccgggcctgctcctaagggagtacctttatggcggcggccgggatgaggagccctccggagcggcccctgagggcggcgcgacccccaccgcggcccccgagacccccgccccgccgacgcgggagacttgctacttcctcaacgccaccatcctattcctgttccgggagttgcgggacaccgcgctgacccgccgctgggtcaccaagaagatcaaggtggagttcgaggagctgctgcagaccaagacggccgggcgcctgctggaggggctgagcctgcgggacgtgttcctgggcgagacggtgcccttcatcaagaccatccggctcgtgcggccagtcgtgccctcggccaccggggagcccgatggccctgaaggggaggcgctgcccgccgcctgccccgaggagctggccttcgaggcggaggtggagtacaacgggggcttccacctggccatcgacgtggacctggtcttcggcaagtccgcctacttgtttgtcaagctgtcccgcgtggtgggaaggctgcgcttggtctttacgcgcgtgcccttcacccactggttcttctccttcgtggaagacccgctgatcgacttcgaggtgcgctcccagtttgaagggcggcccatgccccagctcacctccatcatcgtcaaccagctcaagaagatcatcaagcgcaagcacaccctaccgaattacaagatcagg

SEQ ID NO: 4 - DNA, 263 bp, Sprague Dawley rat
gatcaaggtggagttcgaggagctgctgcagaccaagacggcctttttttttttggaggggctgagcctgcgcgacgtgttcctgggtgacaccgtgccctacatcaagaccatccggctggtgcggcccgtggtggcttcgggcaccggcgagcccgacgaacccgatggggacgctctgcccgccacctgcccgggggagctggcctttgaggcggaggtggagtacaacggcggcttccacctggccatcgacgtggatc

SEQ ID NO: 5 - DNA, 22 bp, Artificial Sequence
  Synthetic Primer
aaggtggagttcgaggagctgc

SEQ ID NO: 6 - DNA, 22 bp, Artificial Sequence
  Synthetic Primer
gtggaagccgccgttgtactcc

(19) United States
(12) Patent Application Publication
(10) Pub. No.: US 2003/0044811 A1
(43) Pub. Date: Mar. 6, 2003

(54) EST-defined probe for cancer progression

(75) Inventor: Randy D. McKinnon, Piscataway, NJ (US)

Correspondence Address: PERKINS COIE LLP, POST OFFICE BOX 1208, SEATTLE, WA, 98111-1208, US

(73) Assignee: University of Medicine & Dentistry of New Jersey

(21) Appl. No.: 10/051,769

(22) Filed: Oct. 20, 2001

Related U.S. Application Data
(60) Provisional application No. 60/242,160, filed on Oct. 20, 2000.

(51) Int. Cl. C12Q 1/68; C07H 21/04; C12P 19/34

(52) U.S. Cl. 435/6; 536/24.3; 435/91.2

(57) ABSTRACT

Nucleic acid sequences that identify a gene product associated with Glioblastoma Multiforme are disclosed. Nucleic acid probes for mRNA transcripts whose expression is associated with glioblast transformation and methods for using these probes in identifying patients at risk for progression into a malignant phenotype are also disclosed.

### CROSS-REFERENCE TO RELATED APPLICATIONS

The present utility patent application claims priority to provisional patent application U.S. Ser. No. 60/242,160 (McKinnon, R. D.), filed Oct. 20, 2000, the disclosure of which is incorporated by reference in its entirety herein.

### GOVERNMENT INTEREST

This work was supported by National Institutes of Health Grant RO1 MH54652. This invention was made with government support. The government may own certain rights in the present invention.

### FIELD OF THE INVENTION

The present invention relates to the field of brain cancer therapy, treatment and diagnosis.

Introduction

Glioblastoma multiforme (GBM), the single most fatal form of cancer known to man, has been termed “The Terminator” (Proc. Natl. Acad. Sci 97:6242-44). It is 95% fatal within 10 months of diagnosis, independent of intervention approaches, and there is a disturbing recent increase in incidence especially in the elderly. The disease amounts a terrible toll on patients, families, and clinicians charged with their care. In spite of immense scrutiny, essentially nothing is known of the etiology, cell physiology and molecular genetics of the disease. In addition, attempts at treating the disease have been unsuccessful due to the complex character of the tumor. Thus, novel therapies and treatments for this disease are important and urgently desired.

Several genetic loci are frequently deleted in GBM tumor cells, implicating gene product(s) whose biochemical actions prevent tumor progression (tumor suppressor genes). One such locus, chromosome 10 band q25, is now completely defined by nucleotide sequence data available in public domain data banks, Accession NT000545 at: (http://www.ncbi.nlm.nih.gov).

A large number of previously described genes and predicted protein encoding regions are located at this chromosomal address. However, specific gene products involved in progression of glioblasts into a malignant phenotype have not been elucidated. Identification of these specific gene products and their function in tumor progression will provide valuable tools in the cancer treatment, therapy and diagnosis.

### SUMMARY OF THE INVENTION

The present invention identifies the exact nucleotide location of a specific gene encoded in 10q25 whose expression is altered during progression from normal glioblasts into immortal glial cells, precursors of a malignant phenotype.

The present invention further relates to an expressed sequence tag (EST) (SEQ ID NO: 2), representing a gene product associated with immortal glioblasts and GBM. In a further aspect of the invention, methods for using the EST as a molecular marker for tumor cell identification and classification are disclosed. Methods for detecting whether a sample from a patient has a propensity for the malignant phenotype are provided. In yet a further aspect of the invention, methods for using the gene product identified by SEQ ID NO: 2 for therapeutic intervention in brain cancer, including glioblastoma multiforme are disclosed.

An additional aspect of the invention relates to kits for use in diagnosing or identifying candidates at risk for progression into a malignant phenotype.

### BRIEF DESCRIPTION OF THE DRAWINGS

FIG. 1. Partial nucleotide sequence of the region of human chromosome 10 encoding GliTEN (SEQ ID NO: 1). The nucleotide sequence is from the NCBI Genbank data files (accession number AC005887). Shown are regions of the human locus identified by homology to a rodent glioblast-derived EST (clone 24.53, 87% identical to underlined sequence (SEQ ID NO: 2)) and the flanking human sequences encoding an open reading frame (capitalized letters (SEQ ID NO: 3)). Double underline: stop codon predicted to lie within intervening (intron) sequences. The encoded protein is 33% and 30% identical to the amino (N)-terminus of proteins predicted from genome sequence analysis of Drosophila and C. elegans. Both fly and worm predicted proteins also encode a carboxy terminus “C1” domain which is highly related (50% amino acid identity) to human chromosome 10 sequences located proximal to the sequence shown.

FIG. 2. Northern blot analysis of GliTEN transcripts in adult rat tissues. Poly(A)-selected mRNA from adult rat tissues were probed with the rat glioblast EST probe 24.53. The probe identifies a large (approximately 7,000 nt) transcript as well as a smaller (approximately 4,000 nt) transcript expressed at high levels in three independently isolated immortal glioblast cells lines (clones 6a, 6b, 7) as well as brain cortex (cx), liver (lv), thymus, and normal rat kidney (NRK) cell line; lower levels were observed in the testes (ts). The blot contains 1 μg mRNA from each tissue, and the exposure time was 16 hours at 70° C.

### DETAILED DESCRIPTION OF THE INVENTION

The present invention relates to an EST (SEQ ID NO: 2), expressed nucleotide sequence tag, representing a gene product associated with GBM. Related embodiments of the invention relate to using the EST as a molecular marker for tumor cell identification and classification, and as a target for therapeutic intervention in glioblastoma multiforme.

The chromosome 10 band q25 locus is frequently deleted in brain tumor cells. The deletion of this locus in tumor cells implies that at least some of the genes encoded in this locus are tumor suppressor genes. The 10q25 locus contains a large number of genes and predicted protein encoding regions which have not been characterized. More specifically, defined genes and gene products involved in the progression of glioblasts into a malignant phenotype have not been disclosed. The present invention identifies the location of a gene in the 10q25 locus that herein is implicated in progression of brain cells into a malignant phenotype.

One embodiment of the invention relates to an EST comprising the sequence disclosed at SEQ ID NO: 2. The EST is associated with a predicted gene product, termed GliTEN, which is in turn associated with glioblastoma multiforme.

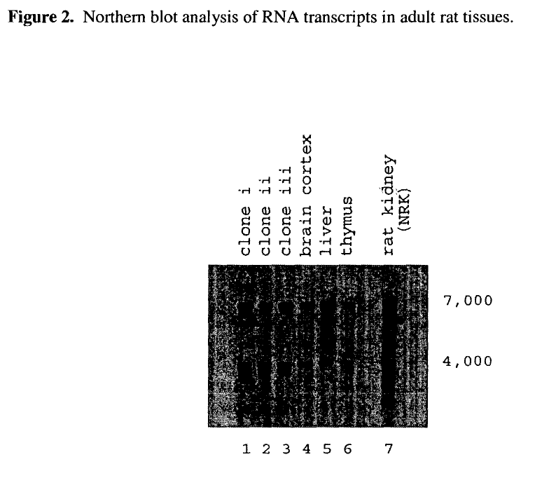

In another embodiment, the present invention is used in the diagnosis of brain cancer. In a preferred embodiment, the present invention is used to diagnose or identify candidates at risk for progression into glioblastoma multiforme. As demonstrated herein, glioblasts express increased levels of nucleic acid associated with SEQ ID NO: 2. Increased levels of these nucleic acids act as a signal to indicate a candidate's risk for progression into the malignant phenotype. The term “increased levels” relates to the steady-state expression of a nucleic acid sequence or encoded protein in a tumor cell with a higher level, preferably at least two-fold higher, than the level observed in a non-tumor cell from normal tissue (control sample or cell). For example, compare FIG. 2 lanes 1, 2, and 3 (immortal glioblasts) with lane 4 (normal adult brain sample).

Hybridization of nucleic acids is typically performed under stringent conditions. The term “stringent conditions” refers to conditions which permit binding of a nucleic acid probe molecule to a highly homologous sequence, and not to non-related sequences, as defined in FIG. 5 of McKinnon et. al., Mol. Cell. Biol. 7:2148-2154, 1987.

The methods for diagnosing or identifying a candidate or patient at risk for progression into the malignant phenotype involve detecting increased levels of SEQ ID NO: 2 associated nucleic acid expression in a sample from a candidate or patient. An example of a relevant sample would be biopsy material from a patient who has a suspected brain tumor such as low grade astrocytoma or oligodendroglioma, which may have the potential in the absence of aggressive therapy to progress into glioblastoma. Methods for detecting increased levels of nucleic acid expression are well known in the art and can include, but are not limited to, nucleic acid hybridization assays such as Northern blot assay, dot blot assay, microarray assays, in situ hybridization assay, polymerase chain reaction and numerous other techniques and assays or combinations thereof (Sambrook and Russel, Molecular Cloning, A Laboratory Manual, Cold Springs Harbor Laboratory Press, N.Y., 2001). Labels for use in the detection techniques and assays include, but are no limited to, fluorescent dye molecules, fluorophores such as fluorescein and fluorescein derivatives, radioactive labels, chemiluminescent labels, or enzyme labels. In one embodiment of the invention, probes comprised of the EST are added to a sample which has been obtained from a candidate or patient and attached to a solid support nylon membrane, and the mixture is incubated then rinsed using standard hybridization protocols. The amount of bound probe is quantified using methods including, but not limited to, autoradiographic detection, and compared to a control sample from normal tissue. Increased levels of expression as compared to normal cells is an indication that the candidate or patient is at risk for progression into a malignant phenotype.

Another embodiment of the invention provides for kits for use in diagnosing and/or identifying candidates at risk for progression into the malignant phenotype. The kits comprise probes specific for GliTEN associated nucleic acids. Preferably, the probes comprise the nucleotide sequence of SEQ ID NO: 2. The kits further comprise reagents and components necessary to perform assays or instructions to practice the methods of this invention.

Another embodiment of the invention provides for kits to use in diagnosing and/or identifying candidates at risk for progression into malignant phenotype. The kits comprise synthetic oligonucleotide probes specific for SEQ ID NO: 2 and GliTEN associated nucleic acids including, aaggtggagttcgaggagctgc (SEQ ID NO: 5), and gtggaagccgccgttgtactcc (SEQ ID NO: 6). The kit further comprises reagents and components necessary to utilize SEQ ID NO: 5 and SEQ ID NO: 6 as primers for polymerase chain reaction (PCR) amplification reaction under standard PCR conditions, to detect the presence and abundance of SEQ ID NO: 2 in RNA isolated from patient material.

### EXAMPLE 1

### Isolation of Rodent Glioblasts and Immortalization In Vitro

Glioblasts were isolated from the rodent brain and maintained in a defined primary cell culture environment in vitro, using the protocols as described (McCarthy and de Vellis, J. Cell Biol 85: 890-902, 1980; Behar et al., J. Neurosci. Res. 21: 168-180, 1988). Glioblasts are obtained from two day old rat brain samples by isolating the cerebral hemispheres, dissociating the tissue by passage through 25-gague needles, then placing the tissue into a culture medium comprised of minimal essential medium supplemented with fetal bovine serum as described (McKinnon et al., Neuron 5, 603-614, 1990). Glioblasts were separated from these cultures by immunoselection (Id.) and placed into fresh culture medium. Under defined culture conditions composed of minimal essential medium supplemented with growth promoting hormones fibroblast growth factor, platelet derived growth factor, and insulin (defined below) these primary glioblasts undergo a spontaneous process of immortalization. The cell culture techniques that facilitate this process are described (Neuron 5, 603-614; J. Neuroscience Research 31:193-204, 1992).

Isolation and culture of primary rat glioblasts is defined as follows. Part 1 was performed on a lab bench with closed door to room to limit air flow, and Parts 2 & 3 were performed in a standard tissue culture hood.

Part 1: Surgical Procedures for Establishing Mixed Rat Brain Glial Cultures

Postnatal day 2 rat pups are decapitated, and pinned nose down on a paper towel on top of styrofoam board using 21 g needle, then the cranial skin is soaked with 70% ethanol. To remove the brain, first lift the skin, cut with small sharp sterile scissors from neck and under ear to one eye, then cut across to second eye to reveal skull. Next, using small scissors, cut down through front of cranium (olfactory bulbs) from midline towards each orbit; repeat at caudal cranium (cerebellum) towards side of neck; next, lift skull with tips of same scissors and cut along midline, then open the skull to reveal forebrain underneath. Remove the brain from cranium with sterile curved forceps and place in 100 mm tissue culture dish (Falcon) with 35 ml MEM Hepes (Gibco Biologicals, Bethesda Md.) plus antibiotics (media should be at 4° C. during this procedure). Repeat dissections of remaining pups, assembly line fashion). Place brains in a dish on a dissecting scope, then use sterile No. 5 forceps to remove meninges: first split the brain longitudinal into 2 halves, then lay one half flat and hold in place with one forcep, pinch the olfactory bulb and lift (meninges should peel off by looking for blood vessels in microscope). Repeat for all brains, moving cleaned ones into dish with fresh MEM Hepes.

Part 2: Cultures

In tissue culture hood, place cleaned brains in 50 ml tube (Falcon) and remove all but 10 ml media. Dissociate using 10 cc syringe, pass through 19 g, 21 g needles (3 times each direction) and finally, 25 g needle (1 time into syringe). Expire through a 25 g needle into sterile 70 um mesh over a 50 ml tube, using Falcon 2350 Cell Strainer (Beckon Dickenson Labware). Centrifuge at 1,000 rpm for 10 minutes. Aspirate media and resuspend pellet in DMEM (high glucose) with 10% fetal bovine sera (10% DMEM). It is best if the sera is thawed at 4° C. and not heat inactivated. The final volume should be 10 ml per 2 brains. Plate 10 ml per flask in Falcon 75 cm2 tissue culture flasks, then place in 37° C. incubator (10% CO2) with caps loose for 3 days; this we term ‘primary cultures’.

Refeed cultures on day 3. Remove media by pipette (save in 50 ml tube), then add fresh media to flasks and return to incubator. Centrifuge the media saved from first refeed of primary culture (1,000 rpm, 10 min.), then aspirate media from the pellet and resuspend the pellet in DMEM 10% FBS (5 ml per original flask). Plate this suspension (10 ml each in T75 Falcon flasks) and return to the incubator; this we term ‘secondary cultures’.

Refeed all cultures on days 6, 9, 12, and 15 with DMEM plus 10% FBS. Flasks generally are confluent by days 5-6.

Part 3: Purification of Primary Glioblasts (day 8; 2 hrs)

When confluent, monolayers (type 1 astrocytes) will have microglia (large, unattached phase bright cells) and glioblast cells (very small, round, blue cells attached to astrocytes monolayer). To remove microglia, place flasks (flat, caps on tight) on rotary shaker (Innova 2000, New Brunswick Scientific) at 37° C. and shake at ˜110 rpm for 2 hrs; remove from the shaker, leave vertical, and place flasks in a cell culture hood, aspirate the media and refeed cells with fresh DMEM plus 10% FBS, then return to CO2 incubator for 4-6 hrs. To detach glioblast progenitors by mitotic shake-off, next place flasks on the rotary shaker at 110 rpm and leave as such for 12-16 hrs. To recover loose cells, collect media from the flasks and save pooled media in a 50 ml tube; refeed the flasks and return to CO2 incubator (glioblasts cells can be harvested 2-3 times for each surgical preparation, and the degree of microglial contamination decreases with each round of purification). Next centrifuge the 50 ml tube with media containing loose cells (1,000 rpm, 10 min), aspirate the media, and resuspend cell pellets in 1.0 ml MEM-Hepes, 0.5% FBS. At this point, glioblast cells can be further purified by one of several techniques as follows.

[A] Culture cells in the presence of mitogens (10 ng/ml PDGF-AA, 5 ng/ml bFGF) for selective amplification of glioblast progenitors. [B] Alternatively, glioblasts can be further purified by removing contaminating cells (principally microglia) by indirect immunopanning. First, incubate cells at room temp for 10-15 min with monoclonal antibody A2B5 (either 1:100 dilution of ascites fluid or 1:10 dilution of tissue culture supernatant, sterilized by filtering through 0.45 uM Costar Spin-X Centrifuge filter units, Costar Cat. No. 8162). After incubation, dilute the cells to 10 mL in 1.0 ml MEM-Hepes, 0.5% FBS then plate cells on 100 mm Falcon dish and incubate at room temp for exactly 7 min with no vibrations. After 7 min, swirl the plate on the lab bench exactly seven times, gently but deliberately, such that non-attached cells are resuspended. Immediately place in culture hood, tip the plate, and collect the media containing the detached cells. Count cells to determine recovery (generally 5×10⁶ pure glioblasts per 15 flasks or 20 animals). [C] Alternatively, glioblasts can be purified by differential adherence. First, plate the cells in 10 ml of culture media on a 10 cm Falcon culture dish, then incubate for 30 min at 37° C.; microglia will adhere, and glioblasts can be recovered by gently swirling to suspend the loose cells.

Centrifuge the cells recovered in step [B] or [C] above, and resuspend at 2×10⁶ cells per ml in 10% DMEM. Plate on poly-ornathine coated coverslips or dishes. For coverslips, pipette 25 ul (5×10⁴ cells) onto 12 mm coverslips (generally 10 coverslips per 60 mm dish); for dishes, pipette 1.0 ml (2×10⁶ cells) onto the center of a 60 mm Falcon dish (drop should cover ˜50% of surface area); carefully move the plated cells into a CO2 incubator. After 30-60 minutes cells should be attached, and in a sterile tissue culture hood add 10% DMEM (5 ml per 60 mm dish; 10 ml per 10 mm dish), then return cells to the CO2 incubator; leave at 37° C. for at least 12 hrs. On day 2 (15-24 hrs. later), refeed the dishes with defined media consisting of Gibco/BRL Dubelcco's MEM, high glucose plus 1 mm Na pyruvate, 25 ug/ml gentamicin, 0.5% FBS, 50 ug/ml transferrin, 25 nM selenium, 30 nM T3, 50 ng/ml bovine insulin.

REAGENTS

(1) Animals: Sprague Dawley rat pups (with mom), 2 days old on arrival. Source: Taconic Farms, N.Y. (2) Equipment: a) A good dissecting microscope, hemocytometer. b) Sterile surgical tools: No. 5 forceps, curved forceps, small & large scissors. c) One each: 12 cc syringe, 19 g, 21 g, 25 g needles. d) Falcon cell strainer (Falcon 2350 Cell 5 Strainer; Beckon Dickenson Labware). e) Spin-X filters (Costar Spin-X Centrifuge filter units, Cat. No. 8162). f) Glass coverslips (Fisher Scientific, 12 mm). (3) Tissue Culture Reagents: a) Tissue culture facility, plastic dishes, culture media. b) Fetal bovine sera (Hyclone, Inc.). Thaw at 4° C., and do not heat inactivate. c) Defined media supplements: [transferrin: Sigma #T2252; 10 mg/ml in PBS, freeze; selenium: Sigma #59133; 3 mM, freeze; tri-ido thyronine (T3): Biofluids #354; 30 mM, freeze; bovine insulin: Sigma #T1882; 10 mg/ml, 4° C., 4.0IN HCL]. d) Poly-L-Lysine (Sigma). (4) Bench top centrifuge (50 ml tubes, 1,000 rpm). (5) Antibodies: A2B5, 04, GC, etc. (6) Growth Factors (Upstate Biotech, Inc.): Basic fibroblast growth factor (human recombinant; stock=1 ug/ml, final=1-5 mg/ml *(replenish every 36-40 hrs). Platelet derived (PDGF) human recombinant PDGF-AA, stock =10 ug/ml, final =10 ng/ml.

### EXAMPLE 2

### Analysis of Glioblast Transformation

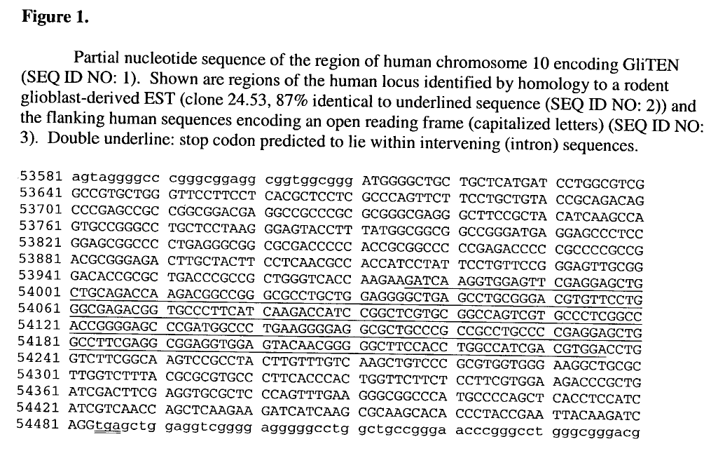

The molecular processes underlying the transformation of primary glioblasts in vitro was examined and the resulting array of expressed nucleic acid (RNA) transcripts was characterized using the published techniques of subtractive hybridization (Representational Difference Analysis, RDA; Nucleic Acids Res. 22:5640-48, 1992). Several (n=155) expressed sequence tags (ESTs) whose mRNA transcripts were maintained at an elevated steady state level in immortal glioblasts were characterized. The characterized ESTs were examined individually by determination of their DNA sequence using standard approaches in a core sequencing facility. The obtained sequences were individually imported into NCBI Blast (http://www.ncbi.nlm.nih.gov/BLAST) to screen for potentially related nucleic acid sequences in public domain databases. One EST transcript (clone number 24.53) (SEQ ID NO: 4), gat caaggtggag ttcgaggagc tgctgcagac caagacggcc tttttttttt tggaggggct gagcctgcgc gacgtgttcc tgggtgacac cgtgccctac atcaagacca tccggctggt gcggcccgtg gtggcttcgg gcaccggcga gcccgacgaa cccgatgggg acgctctgcc cgccacctgc ccgggggagc tggcctttga ggcggaggtg gagtacaacg gcggcttcca cctggccatc gacgtggatc, represents a glioblast EST that maps to human chromosome 10 band q25 (Genbank accession AC005887) with 86% nucleic acid sequence identity, confirming that the mRNA transcript of cDNA 24.53 is the rat homologue of a human mRNA transcript (FIG. 1).

### EXAMPLE 3

### Northern blot Analysis of GliTEN Transcripts

Total cell RNA is isolated from tissue samples using commercially available reagents and procedures described therein (Gibco Trizol), obtained from animal organs, from animal cells in culture, or from patients at the time of surgical biopsy or tumor resection. Poly(A)-selected mRNA from adult rat tissues were probed with rat glioblast EST probe 24.53. The blot contained 1 μg mRNA from each tissue and the exposure time was 16 hours at 70° C. Examination of mRNA transcripts revealed hybridization to two transcripts, approximately 7,000 and 4,000 nucleotides in length, expressed at high levels in three independently derived immortal glioblast cell lines in vitro and in several adult tissues including brain and liver (FIG. 2). Analysis of cDNA generated from these same RNA samples by reverse transcriptase-polymerase chain reaction (RT-PCR) further confirmed the presence of a cognate of this transcript in human brain. For PCR, RNA is reverse transcribed into single stranded cDNA using oligo(dT) primer using commercially available kits (Gibco BRL) for cDNA synthesis and procedures described therein. PCR analysis was performed using 100 ng template cDNA in a 50 ul reaction consisting of 0.25 uM synthetic oligodeoxynucleotide primers (SEQ ID NO: 5, SEQ ID NO: 6), 0.1 mM dNTP's, 2.5 mM MgCl2, 5 units Taq polymerase (Gibco/BRL, Bethesda Md.) and Taq reaction buffer supplied by the manufacturer. The primers, SEQ ID NO: 5 and SEQ ID NO: 6, for amplification of the SEQ ID NO: 2 (GliTEN transcripts) were obtained from commercial vendors (IDT, Coralville Iowa). PCR amplification was performed using a Perkin-Elmer thermocycler with 30 cycles [95° C., 1 min; 58° C., 2 min; 72° C., 3 min] followed by 10 min at 72° C. extension. The PCR products were separated on 1.5% agarose gels containing 0.5 ug/ml ethidium bromide, and DNA products were visualized by UV trans-illumination. All electrophoretic analysis included a DNA mobility marker (HaeIII digest of psi-X174 DNA, Gibco/BRL), and PCR products were identified by relative electrophoretic mobility.

### EXAMPLE 4

### GliTEN, Gene Product located on Human Chromosome 10 band g25

Examination of the human 10q25 chromosomal locus (FIG. 1) revealed a 892 base pair (bp) region flanking the original EST with a single open reading frame for protein translation (AC005887, nucleotide positions 53,611-54,483 inclusive). This predicted protein is herein referred to as GliTEN, since the original rodent EST (clone 24.53) was identified in immortal Glioblasts and since the rodent EST (clone 24.53) maps to human chromosome ten, a locus whose mutation is associated with glioblast transformation.

A sequence alignment search for proteins related to this predicted protein using the NCBI Genbank ‘tblast’ algorithm revealed two highly homologous proteins predicted to be encoded in the genomes of Drosophila melanogaster (CG10362, Genbank accession AAF48119) and C. elegans (Genbank accession CAB54213). The D. melanogaster sequence has been detected as ESTs (clone numbers CK2546, LD34222) expressed in the embryonic brain, as reported by the Berkeley Drosophila Genome Project (web address: http://www.fruitfly.org;). Neither the fly or worm homologue has been further characterized, and to date these molecules are defined only as ‘theoretical’ gene products.

In summary, analysis of transcripts that were elevated in the process of rodent glioblast immortalization led to the identification of an mRNA transcript that had not been previously characterized in any organism. The human homologue of this transcript was then mapped to human chromosome 10q25, which is associated with brain cancer, and a predicted protein, GliTEN, was determined and implicated in the process of glioblast transformation and turmorogenesis.

### EXAMPLE 5

### Methods for Detecting Candidates at Risk for Progression into GBM

The nucleotide sequence herein referred to as EST (SEQ ID NO: 2), encoding a portion of the predicted gene product GliTEN, is a molecular probe for a mRNA transcript whose expression is associated with glioblast transformation. SEQ ID NO: 2 serves as a probe for characterizing glioblast tumors in humans, with specific emphasis on its use in identification of tumors which are likely candidates for progression into glioblastoma multiforme.

The probe defined herein as SEQ ID NO: 2 represents a molecular marker for determining the abundance of RNA transcripts of this sequence present in normal, immortal, and pre-malignant cells. The abundance of these RNA sequences is determined by methods including but not limited to RNA blot analysis, using SEQ ID NO: 2 as a molecular identifier for the presence of such RNA transcripts, or PCR amplification, using SEQ ID NO: 5 and SEQ ID NO: 6. Samples to be examined by this analysis are obtained from patients by surgical resection, such as but not limited to surgical biopsy material and surgical specimens removed from a patient at the time of surgical resection to debulk an existing tumor.

Samples are immediately processed for the isolation of total cell RNA molecules from this tissue using the Trizol reagent and protocols as detailed by the reagent manufacturer (Gibco BRL), these representing standard protocols for the isolation of total cell RNA from any source of tissue. Blot analysis is defined as the fractionation of a sample of said tissue RNA (5-10 micrograms is generally sufficient) on an agarose gel containing formaldehyde, with adjacent lanes containing appropriate control tissue samples, test samples, and molecular weight markers, as described in McKinnon et al (Neuron 5, 603-614, 1990).

The samples are then transferred to nylon membranes and processed for hybridization analysis with SEQ ID NO: 2 labeled probe using standard conditions as described in Sambrook and Russel (Molecular Cloning, a laboratory manual, 3rd Edition; Cold Spring Harbor Laboratory Press, 2001). In the case of p³² labeled radioactive probes, the RNA transcripts hybridizing to SEQ ID NO: 2 are visualized, after probing and subsequent washing of the blot to high stringency, by exposing the nylon membrane to an emulsion film (Fuji RX medical X-ray film) and developing the resulting autoradiographic exposure. Control samples include, but are not limited to, RNA isolated from non-cancerous ‘normal’ tissue obtained during the procedure that generated the suspected or known tumor specimen, RNA isolated from human cell lines with characteristics similar to those of the cancerous lesion (human tumor cell lines are commercially available in public repositories such as American Type Culture Collection, Rockville MD), RNA isolated from normal rat brain glioblasts, and RNA isolated from immortal rat brain glioblasts.

The blot analysis of transcripts expressed in a patient's sample will identify a 7,000 nucleotide and a 4,000 nucleotide RNA, containing sequences complementary to the probe SEQ ID NO: 2, that represent the bona fide messenger RNA encoding the GliTEN protein. PCR analysis of SEQ ID NO: 2 expression in such samples would be undertaken after reverse transcription of such RNA samples, and subsequent PCR amplification using the SEQ ID NO: 2 specific primers, SEQ ID NO: 5 and SEQ ID NO: 6, as outlined in Example 3 above. The results of this analysis will reveal the level of expression of these specific RNA transcripts in the patient samples, and will allow a determination of their level of expression in those samples relative to normal tissue, non-cancerous tissue, and cancerous tissue.

An elevated level of expression, detected as a specific elevation in the intensity of autoradiographic signal of SEQ ID NO: 2 transcripts, is observed in immortal rat glioblasts relative to their levels in normal primary culture rat glioblasts. A similar elevated level of SEQ ID NO: 2 transcripts in a surgical biopsy from a suspected brain lesion, relative to the level of SEQ ID NO: 2 transcripts in adjacent normal tissue, is taken as evidence that the suspected lesion site contains cells which have the potential to progress into glioblastoma multiforme. Such evidence gives reasonable grounds for the need to pursue an aggressive clinical strategy to eliminate such lesions from the patient.

### EXAMPLE 6

### Method for characterizing GliTEN

SEQ ID NO: 2 represents a short segment of a large (7,000 nucleotide) RNA transcript expressed in immortal glioblasts. The protein GliTEN encoded within this sequence, based on homology between human, Drosophila and C. elegans genomic sequences, is predicted to have a molecular size of 114,554 kilodaltons encoded in approximately 4,500 nucleotides of this transcript. The full length cDNA encoding GliTEN is obtained from normal glial cells by selective PCR amplification of the transcript, using standard molecular biological procedures. RNA from tissues containing SEQ ID NO: 2 transcripts is isolated and reverse transcribed into first strand cDNA as described in Example 5, then PCR amplified using sets of oligodeoxynucleotide primers including SEQ ID NO: 5 and SEQ ID NO: 6.

To isolate sequences from the 5′ portion of the molecule, PCR reactions are carried out using a commercial kit (InVitroGen) employing the 5′-RACE protocol. To isolate sequences from the 3′ portion of the molecule, PCR reactions are carried out using SEQ ID NO: 5 and the 3′-primer oligo(dT). PCR products are amplified using standard thermocycling conditions, and the products obtained are identified by direct DNA sequence analysis from a Core sequencing facility. The respective 5′ and 3′ sections of the complete cDNA are assembled in a plasmid vector and amplified using standard bacteriological cloning as described in Sambrook and Russel (Molecular Cloning, a Laboratory Manual, 3rd Edition; Cold Spring Harbor Laboratory Press, 2001).

Based on these findings, it is believed that GliTEN will be useful in therapy and treatment of brain cancers, including GBM, since its delivery into glioblastoma tumor cells may suppress the malignant phenotype in patients. The encoded gene product GliTEN may be used as a tumor suppressor in preventing glioblast transformation, and thus the GliTEN transcript may be used in methods for treating GBM, including gene therapy.

Accordingly, further embodiments of the invention involve vectors for use in cancer treatment, comprising a viral or plasmid vector encoding a promoter linked to a GliTEN expression cassette. In a further embodiment, the vectors of this invention may be used in gene therapy approaches to treat cancer, including glioblastoma multiforme. The gene therapy techniques are employed to increase expression of the GliTEN gene in tumor cells, whereby increased expression of GliTEN may suppress tumor growth. Gene therapy techniques allow an absent gene to be replaced with a functional gene. This invention allows for the replacement of an absent gene, which is believed to encode a tumor suppressor protein located in 10q25, with a functional gene. Gene therapy techniques also allow for the delivery and controlled expression of therapeutic gene products. In a further embodiment, the vector containing the GliTEN expression cassette is delivered to the tumor, such as glioblastoma multiforme. The gene therapy techniques may employ adenoviral vectors, adeno-associated viral vectors, recombination-defective retroviral vectors or plasmid DNA vectors to deliver the GliTEN expression cassette into the tumor or cancerous cells. The vectors of this invention may be used to increase GliTEN levels within tumor cells and thereby suppress tumor growth.

The term “vector” refers to a nucleic acid construct engineered to encode a particular gene product. The vectors of the present invention can include adenoviral, adeno-associated viral, recombination-defective retroviral, or plasmid DNA vectors. The vectors include all necessary sequences for the expression of the GliTEN expression cassette and any sequences that may be included to control the expression of the cassette. These sequences may include, but are not limited to, a promoter or initiation sequence, an enhancer sequence, termination sequence, RNA processing signals, and/or a polyadenylation signal sequence.

The term “GliTEN expression cassette” refers to nucleic acid which codes for the GliTEN protein product as defined in Example 6. Due to the degeneracy of the genetic code, a number of nucleic acid sequences that encode the GliTEN protein product may be produced. A number of these sequences will only have minimal homology to the naturally occurring GliTEN nucleic acid sequence. Each nucleic acid sequence variation based on the various possible codon choices is contemplated by this invention. The expression cassette is positioned within the vector such that it can be transcribed into RNA and translated into the GliTEN protein product.

The term “necessary sequences for the expression of GliTEN” refers to sequences required to ensure the RNA transcription and subsequent translation of the expression cassette to produce GliTEN polypeptide sequences. The term “promoter” refers to a DNA sequence that is bound by RNA polymerase and is required to initiate RNA transcription of a gene. There are a number of promoters that are known in the art, including those that can enhance or control expression of the gene or expression cassette. For example, cytomegalovirus early promoter may be fused to the GliTEN expression cassette to obtain constitutive expression of the cassette.

The vectors of this invention may be delivered directly to the location of the tumor cells by injection. The vectors may be administered or delivered in saline solutions or encapsulated in liposomes. Delivery into the area of the tumor is performed at the time of biopsy or after a surgical debulking procedure. The term “tumor” refers to cancerous cells, including those with a malignant phenotype, such as glioblastoma multiforme.

One skilled in the art will readily appreciate that the present invention is well adapted to carry out the objects and obtain the ends and advantages mentioned as well as those inherent therein. The nucleic acid sequences along with the methods and procedures described herein are presently representative of preferred embodiments and are exemplary and not intended as limitations on the scope of the invention. Changes therein and other uses will occur to those skilled in the art which are encompassed within the spirit of the invention or defined by this scope with the claims.

It will be readily apparent to one skilled in the art that varying substitutions and modifications may be made to the invention disclosed herein without departing from the scope and spirit of the invention.

All patents and publications referenced herein are incorporated by reference to the same extent as if each individual publication was specifically and individually indicated to be incorporated by reference.

What is claimed is:

1. An isolated nucleic acid comprising the nucleotide sequence of SEQ ID NO: 2.

2. An isolated nucleic acid comprising a sequence that hybridizes under stringent conditions to a nucleic acid comprising a nucleotide sequence of SEQ ID NO: 2, or the complement thereof.

3. A probe for use in identifying a patient at risk for progression into the malignant phenotype comprising the nucleotide sequence of SEQ ID NO: 2, or the complement thereof, or the labeled nucleotide sequence of SEQ ID NO: 2, or the complement thereof.

4. The probe of claim 3, wherein the label is a fluorescent dye molecule, a radioisotope, a chemiluminescent molecule, or an enzyme.

5. A method for detecting whether a patient is at risk for progression into glioblastoma multiforme comprising,
a) providing a sample from a patient;
b) adding a labeled probe comprising the nucleotide sequence of SEQ ID NO: 2 to the sample or performing PCR analysis using SEQ ID NO: 5 and SEQ ID NO: 6;
c) analyzing levels of mRNA bound with the probe;
d) treating a control sample according to the method to assess the level of mRNA in a control sample; wherein the presence of increased levels of mRNA expression in the sample in an amount higher than the control sample indicates risk for progression into glioblastoma multiforme.

6. A kit for use in detecting whether a patient is at risk for progression into glioblastoma multiforme comprising nucleotide sequence probes of SEQ ID NO: 2 and instructions for use.

7. The kit of claim 6, further comprising reagents and components for use in performing assays.

8. A kit for use in detecting whether a patient is at risk for progression into glioblastoma multiforme comprising nucleotide sequence probes of SEQ ID NO: 5 and SEQ ID NO: 6 and instructions for use.

9. The kit of claim 8, further comprising reagents and components necessary to use SEQ ID NO: 5 and SEQ ID NO: 6 as primers for PCR amplification reaction.
